(19) United States
(12) Patent Application Publication
(10) Pub. No.: US 2025/0014581 A1
(43) Pub. Date: Jan. 9, 2025

(54) SILENT INTRAORAL SPEECH SENSING WITH AUDIO FEEDBACK

(71) Applicant: Augmental Technologies Inc., San Francisco, CA (US)

(72) Inventors: Tomas Alfonso Vega Galvez, San Francisco, CA (US); Corten Singer, San Francisco, CA (US)

(73) Assignee: Augmental Technologies Inc., San Francisco, CA (US)

(21) Appl. No.: 18/888,237

(22) Filed: Sep. 18, 2024

Related U.S. Application Data
(63) Continuation-in-part of application No. 18/099,288, filed on Jan. 20, 2023.
Application No. 18/099,288 is a continuation-in-part of application No. 17/366,186, filed on Jul. 2, 2021, now Pat. No. 11,941,161.
(60) Provisional application No. 63/539,130, filed on Sep. 19, 2023.
(60) Provisional application No. 63/301,501, filed on Jan. 21, 2022.
(60) Provisional application No. 63/162,444, filed on Mar. 17, 2021.
(60) Provisional application No. 63/063,455, filed on Aug. 10, 2020.
(60) Provisional application No. 63/047,946, filed on Jul. 3, 2020.

(51) Int. Cl. G10L 15/25; G01C 21/16; G10L 15/06; G10L 15/22; H02J 50/10; H04R 1/02; H04R 3/00

(52) U.S. Cl. CPC G10L 15/25; G01C 21/16; G10L 15/063; G10L 15/22; H04R 1/025; H04R 3/00; G10L 2015/225; H02J 50/10; H04R 2420/07

(57) ABSTRACT

A processor-implemented method for speech sensing is disclosed. A tongue position sensor (TPS) is embedded palatally in an oral cavity of a person. The TPS detects tongue movement of the person. A barometric sensor and an inertial measurement unit (IMU) sensor are both located in the oral cavity of the person and coupled to the TPS. The TPS, barometric sensor, and IMU sensor together form an intraoral mouthpad that can detect tongue movements, jaw and/or head movements, and breathing of the person. Input from the mouthpad can be analyzed on a processor coupled to the mouthpad. The processor and the mouthpad combine to form an intraoral speech sensing complex. The complex can sense human speech based on the processor output. The complex can sense normal, low-volume, or silent speech of the person. The complex can provide non-blocking audio feedback to the person through a bone conduction speaker.

### RELATED APPLICATIONS

This application claims the benefit of U.S. provisional patent application “Silent Intraoral Speech Sensing With Audio Feedback” Ser. No. 63/539,130, filed Sep. 19, 2023.

This application is also a continuation-in-part of U.S. patent application “Intraoral Electronic Sensing for Health Monitoring” Ser. No. 18/099,288, filed Jan. 20, 2023, which claims the benefit of U.S. provisional patent application “Intraoral Electronic Sensing for Health Monitoring” Ser. No. 63/301,501, filed Jan. 21, 2022.

The U.S. patent application “Intraoral Electronic Sensing for Health Monitoring” Ser. No. 18/099,288, filed Jan. 20, 2023 is also a continuation-in-part of U.S. patent application “Data Manipulation Using Remote Augmented Sensing” Ser. No. 17/366,186, filed Jul. 2, 2021, which claims the benefit of U.S. provisional patent applications “Data Manipulation Using Remote Augmented Sensing” Ser. No. 63/047,946, filed Jul. 3, 2020, “Gestural Sensing Using In-Ear Inertial Measurements” Ser. No. 63/063,455, filed Aug. 10, 2020, and “Intraoral Connected Processing Devices” Ser. No. 63/162,444, filed Mar. 17, 2021.

Each of the foregoing applications is hereby incorporated by reference in its entirety.

### FIELD OF ART

This application relates generally to speech sensing and more particularly to silent intraoral speech sensing with audio feedback.

### BACKGROUND

Modern technology has changed the way people interact with one another and the world around them. The adage “all machines are amplifiers” has been proven by the ever-expanding uses of electronic hardware and computer applications to communicate and interact with other people and things near and far. Personal computers, laptops, pads, tablets, and cellphones allow users to talk, text, and share video information nearly anywhere in the world. Video game competitors can play games with hundreds of others or against computer systems at any time. Workers can access corporate, government, public, or private networks from any place where a phone line or wireless network can be found. Networked hardware can be controlled by users half a world away. Wireless devices can be used for engaging in educational tasks such as classes, tutorials, recitations, and laboratories; performing research tasks such as data collection and analysis; among myriad other uses. Information access can include a wide variety of online content such as politics, news updates, sports scores, and other items of import, interest, amusement, and diversion. Other uses include accessing video streams such as TV programs, movies, adorable puppy and kitten videos, children doing silly things videos, and streaming provided by internet influencers, with each stream intended to provide entertainment and occasionally useful information to the user. Cellphones and tablets are used for keeping in touch with family, friends, coworkers, and other people through email, chat, social media, photos, and even telephony. The ways by which a user employs an electronic device to consume media or to engage with others depend on the particular device. Smartphones are incredibly portable, enabling usage while a person is out and about, traveling, or staying in for a quiet night at home. A smartphone can access the Internet; connect to news, information, and social media sites; enable online shopping; and support email, chatting, and calls; among seemingly countless other uses.

Along with the case of access and portability has come a host of challenges. Texting while driving has become a problem across many countries. Distracted driving has led to accidents, injuries, and deaths in every nation with cellphones and wireless access. Learning processes and preferences have been altered by the vastly expanded availability of video and audio files. Educators are challenged to find new ways to engage students and promote learning. Employers are rethinking the best way to accomplish projects and maintain profits. Remote access workers are forcing private and public organizations to approach management and operations processes differently as resources are spread across different time zones and have shifting attitudes toward time spent in an office. Communicating effectively with more diverse teams of individuals who are not physically in the same space can present challenges to management and team members alike. Data security has become a major concern as organized criminal organizations and determined individuals find ways to disrupt networks, steal identities, or hold vital data for ransom. “All machines are amplifiers” can be both positively and negatively applied. These challenges are not unsolvable, but they will take considered, sustained effort to evaluate and address as our global society continues to leverage the electronic advantages now available.

### SUMMARY

Wireless electronic devices have revolutionized our ability to communicate, access information, complete tasks, and play games. Our cellphones, tablets, and computers allow us to complete many tasks more quickly and easily than ever before. However, overt interaction by a person (user) with a computing or communication device is not always socially unacceptable. At times, such interactions can become impossible, dangerous, or even illegal. In some situations, people are engaging in activities that require them to interact with processors while they are performing other tasks. The tasks can engage the user's hands, thus preventing the user from engaging with common input/output devices. In other instances, the user may have physical challenges or limitations which prevent human-machine interactions. The requirements of disparate tasks can include accessing a repair manual while working on a piece of equipment, reading design specifications while operating a machine, or even using augmented reality while performing surgery. In addition, situations exist in which a person cannot use conventional input/output devices or techniques.

A processor-implemented method for speech sensing is disclosed. A tongue position sensor (TPS) is embedded palatally in an oral cavity of a person. The TPS detects tongue movement of the person. A barometric sensor and an inertial measurement unit (IMU) sensor are both located in the oral cavity of the person and coupled to the TPS. The TPS, barometric sensor, and IMU sensor together form an intraoral mouthpad that can detect tongue movements, jaw and/or head movements, and breathing of the person. Input from the mouthpad can be analyzed on a processor coupled to the mouthpad. The processor and the mouthpad combine to form an intraoral speech sensing complex. The complex can sense human speech based on the processor output. The complex can sense normal, low-volume, or silent speech of the person. The complex can provide non-blocking audio feedback to the person through a bone conduction speaker.

A processor-implemented method for speech sensing is disclosed comprising: embedding a tongue position sensor (TPS) in an oral cavity of a person, wherein the TPS is embedded palatally in the oral cavity, and wherein the TPS detects tongue movement of the person; coupling a barometric sensor and an inertial measurement unit (IMU) sensor to the TPS, wherein the barometric sensor and the IMU sensor are both located in the oral cavity of the person, wherein the TPS, the barometric sensor, and the IMU sensor comprise an intraoral mouthpad, and wherein the mouthpad detects tongue movements, jaw and/or head movements, and breathing of the person; analyzing input from the mouthpad, wherein the analyzing is performed on a processor coupled to the mouthpad, and wherein the processor and the mouthpad comprise an intraoral speech sensing complex; and sensing speech, based on an output of the processor. Some embodiments comprise providing audio feedback to the person, based on the analyzing. Some embodiments comprise coupling a microphone to the intraoral speech sensing complex. In embodiments, the coupling of the microphone is performed by the IMU sensor. In embodiments, the microphone augments output from the barometric sensor for higher accuracy pressure change determination.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for silent intraoral speech sensing with audio feedback.

FIG. 2 is a flow diagram for intraoral speech sensing usage.

FIG. 3 is a system block diagram for silent intraoral speech sensing.

FIG. 4 illustrates system components for silent intraoral speech sensing.

FIG. 5 is a flow diagram for machine learning.

FIG. 6 is a flow diagram for machine learning training.

FIG. 7 is a system diagram for silent intraoral speech sensing with audio feedback.

### DETAILED DESCRIPTION

Users interact with electronic devices in many different ways. Input devices such as keyboards, touchpads, mice, laser pointers, and so on have been used for many years. As the use of networked and wireless devices has grown and diversified, the need to multitask while interacting with devices has grown as well. While voice command and natural language processing have improved the ability to interact with electronic devices and systems verbally, the spoken word is not always appropriate or even legal in some contexts. One or more impairments can be associated with a particular person or user. Motor impairments can prevent a user from typing and controlling a mouse or trackpad, visual impairments can prevent a user from reading a display, and so on. Situational impairments can impede or prevent a user from employing common input/output techniques. Examples include a bright light environment in which a user cannot read a display, a low light situation preventing use of a keyboard, and the like. Situational impairments can also be based on circumstances of the user for which speaking violates rules or social mores, accessing a screen is deemed rude, and the like. Other situations can include the user being engaged in another activity such as machine operation or surgery. In these latter cases, the user is unable to access a keyboard, mouse, or trackpad because the user's hands are otherwise occupied.

A group of sensors can be embedded in the oral cavity of a person, including a tongue position sensor (TPS), one or more inertial measurement units (IMUs), one or more microphones, and a barometric sensor. Together, these sensors can work with a processor to form a mouthpad situated in the upper portion of the mouth. An internal battery and power management system is incorporated into the mouthpad, along with a wireless communications system and bone conduction speaker. The complete set of sensors, microphones, power supplies, wireless communications, and processing constitutes an intraoral speech sensing complex. The processor can access a machine learning model that can recognize words spoken by the user, even when the words are spoken with low-volume or silently. Based on the sensor data and microphone input, each word can be captured, analyzed, and fed back to the user via the speaker. The wireless communications link can send the words spoken by the user to an external device such as a cellphone, computer, or other networked device. The mouthpad can include touchpoints and switches to allow the user the ability to transmit commands, control speaker volume, correct words misidentified by the processor and so on. The result is a wireless link between the user and other wireless devices that can be controlled by voice and/or tongue commands to an intraoral device. The wireless link can be voice activated at any volume level, from a shout to complete silence. Thus, control over a cellphone or other wirelessly linked device can be gained while the user is in an environment where speech would be inappropriate or illegal. Multitasking can be accomplished as the user employs hands, arms, and so on while controlling a wireless device with his or her voice.

Techniques for speech sensing are disclosed. A set of sensors embedded in a retainer-like arrangement can be placed in the oral cavity of a person. The sensors include a tongue position sensor (TPS) which can detect tongue movement within the mouth in three dimensions; one or more inertial measurement unit sensor (IMUs) which can be used to detect tongue or jaw positions, rotation, acceleration, and so on; and a barometric sensor, which can sense ambient barometric pressure, increased barometric pressure due to exhaling into a closed oral cavity, decreased barometric pressure due to inhaling from a closed oral cavity, and so on. These sensors can be combined with a processor which can receive input from the sensors and analyze the data they provide. The analysis can be completed using a machine learning model that can match mouth and jaw movement, tongue movement, and breathing to words recorded in the model's database. Microphones can be included to add sound data to the machine learning model as well. Together, these sensors, microphones, and processors form a mouthpad, which is similar to a mousepad or touchpad on a laptop or tablet. Rather than a hand or finger controlling the input to the pad, the tongue provides the human input to the mouthpad. The mouthpad can include internal power, wireless communications, and a bone conduction speaker that can allow the user to hear the words which the processor identifies as it takes in sensor and microphone data and analyzes it using the machine learning model. The entire system becomes an intraoral speech sensing complex which can identify words and phrases generated by the user whether the mouth speaks the words out loud, at a whisper, or silently.

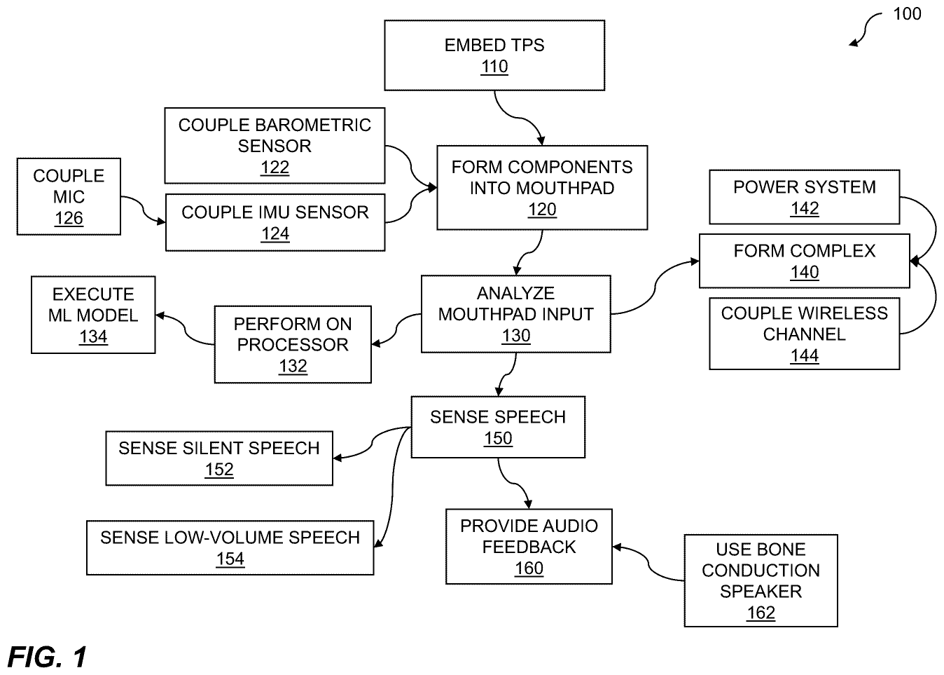

FIG. 1 is a flow diagram for silent intraoral speech sensing with audio feedback. The flow 100 includes embedding a tongue position sensor (TPS) 110 in an oral cavity of a person, wherein the TPS is embedded palatally in the oral cavity, and wherein the TPS detects tongue movement of the person. The tongue position sensor can be located such that it can be easily accessible to the tongue. In embodiments, the TPS can be above the tongue, such as when a retainer is worn on the upper teeth. In other embodiments, the TPS be placed below the tongue of the person (user), such as when a retainer is worn on the lower teeth. The TPS can include electrodes, pressure sensors, barometric sensors, optical sensors, ultrasonic sensors, etc. Tongue input data can be detected by the TPS and can augment, control, or modify data collected by, or being processed by, a processor or preprocessor. The tongue input data can be sensed based on tongue position, tongue pressure, tongue movement, tongue movement direction, tongue movement speed, tongue movement acceleration, and so on. The TPS can comprise a capacitive sensor that not only tracks two-dimension (2D) tongue movement (i.e., roughly in an x-y plane—the TPS conforms to the shape of the upper palate and is therefore not a flat x-y plane, per se), but it can track three-dimension (3D) tongue movement (i.e., adding a z dimension) by determining a lighter or heavier capacitive impingement of the tongue over the TPS surface (i.e., not even necessarily touching the actual TPS surface).

The flow 100 includes coupling a barometric sensor 122 and an inertial measurement unit (IMU) sensor 124 to the TPS, wherein the barometric sensor and the IMU sensor are both located in the oral cavity of the person, wherein the TPS, the barometric sensor, and the IMU sensor form an intraoral mouthpad 120, and wherein the mouthpad detects tongue movements, jaw and/or head movements, and breathing of the person. In embodiments, the barometric sensor can be used to detect ambient barometric pressure, changes in barometric pressure such as increases or decreases, and so on. The barometric sensor can detect a tri-value state of air pressure within an oral cavity containing the interface. The tri-value state can include a number, a code, a percentage, text, and the like. The tri-value state can be sensed from a continuum of pressures and can include ambient barometric pressure, increased barometric pressure due to exhaling into a closed oral cavity, and decreased barometric pressure due to inhaling from a closed oral cavity. The barometric sensor 122 can detect a plurality of pressures along an analog or continuum of possible pressures for sensing.

In embodiments, the IMU can be used to detect tongue or jaw positions, rotation, acceleration, and so on. The inertial measurement unit can be placed at various positions within the mouth. In some embodiments, at least two inertial measurement units are positioned in nonadjacent locations within the mouth. An example placement of the IMUs is where the IMUs are located to the left and the right of a retainer. In other embodiments, the inertial measurement unit is adjacent to the palate of the mouth. The positioning of the IMU can be determined based on efficacy, comfort, convenience, and so on. In embodiments, the IMU can be adjacent to a gumline of the mouth.

The flow 100 further comprises coupling a microphone 126 to the mouthpad. The microphone can comprise a bone conduction microphone. In embodiments, the bone conduction microphone can include a pickup, a transducer, an accelerometer, or another device that can be used to collect audio data within the oral cavity through a mechanical coupling to the upper or lower jawbone, one or more teeth, thin soft tissue covering an oral cavity bone such as the gum, and so on. The bone conduction microphone can comprise micro-electromechanical systems (MEMS) technology. In addition, an air pressure audio microphone can also be coupled to the intraoral speech sensing complex. This microphone can collect ambient sounds, speech, human-generated sounds, and so on. In embodiments, the air pressure microphone enables normal and low-volume 154 speech sensing. The microphone can be used while the person is in a situation where they are only able or permitted to speak very softly, such as whispering in a library. In embodiments, the microphone can be enabled based on an output from an interface-embedded sensor, such as the TPS, the one or more IMUs, and/or other embedded sensors. The microphone can be operated using a “normally off” technique where the microphone can be enabled or turned on based on the TPS, IMU, or barometric sensors. In other embodiments, the microphone can be enabled based on an output from another interface-embedded sensor. In some embodiments, completely silent speech 152 can be sensed by identifying tongue and/or mouth movements to map words, symbols, or letters to be processed by the disclosed method.

The flow 100 includes analyzing input 130 from the mouthpad, wherein the analyzing is performed on a processor 132 coupled to the mouthpad, and wherein the processor and the mouthpad comprise an intraoral speech sensing complex 140. In embodiments, the mouthpad includes data from the TPS, the barometric sensor, the IMU, and the microphone. The processor 132 analyzes the data from the mouthpad to sense and interpret speech generated by the person. The speech can be normal volume, low-volume, or silent speech. Normal speech can be sensed by the microphone. Low-volume and silent speech can be sensed by the data from the microphone, the TPS, the IMU, and the barometric sensor. In embodiments, data from all sensors included in the mouthpad are used to analyze and interpret the speech generated by the person. The position of the tongue, mouth, and jaw can be used to clarify and validate the words spoken by the person. The incoming and outgoing breath of the person, and the pressures generated by the air flowing in and out of the mouth can be used to analyze the words, inflections, accent, intensity, and so on of the speech being generated.

In embodiments, the processor executes code on a machine learning model 134. The executing can include training the machine learning model, discussed later. The training is based on prescribed usage of the intraoral speech sensing complex by the user. The training is performed on a pre-trained machine learning model. The pre-trained machine learning model is from a library of pre-trained machine learning models. The library of pre-trained machine learning models includes models for language, accent, dialect, region, and/or speech impediments. In embodiments, the machine learning model can be trained by recording and analyzing normal speech generated by the person, as well as data recorded from other persons stored in a training library. A set of predetermined words and phrases can be generated by the person and used in the training data library for the machine learning model. The normal speech data collected from the person can be compared to data collected as the person generates the same set of words and phrases using low-volume and silent speech. In some embodiments, the person can participate in training the machine learning model by viewing the text of the low-volume or silent speech analyzed by the processor and validating and/or correcting the text, thereby updating the machine learning model database. In other embodiments, the person can listen to the words analyzed by the processor through a bone conduction speaker 162 as the person generates low-volume and/or silent speech. In addition to low-volume and/or silent speech, audio feedback can be provided via the bone conduction speaker and can include any audio-renderable signal that comes from a connected phone or computing system, which can include a voice call, a text, an email, a summary, directions, an alert, a response from an artificial intelligence large language model agent, and so on. In addition, the person can validate and/or correct the machine learning model using the mouthpad or an external interface, such as a wireless phone, tablet, laptop, or computer linked to the mouthpad (see below).

The flow 100 further comprises powering the intraoral speech sensing complex 140 using a wireless power system 142. In embodiments, the wireless power system comprises a battery, an induction coil, a battery charger, a power regulator, and a Hall sensor. The battery, an induction coil, power regulator, and Hall sensor can be included in the intraoral speech sensing complex. The battery can be charged using a battery charger to induce voltage in the induction coil. The battery charger also includes an induction coil. When the induction coil in the charger is brought into proximity to the induction coil in the complex, the coil in the complex receives induced current. The Hall sensor can be used to sense when the intraoral speech sensing complex has been placed in or near the battery charger and allow the induced current to flow into and charge the battery. The power regulator can be used to maintain voltage from the battery to the various components of the intraoral speech sensing complex.

The flow 100 further comprises coupling a wireless communication channel 144 to the intraoral speech sensing complex. The wireless connectivity can be based on communications standards, preferred protocols, low power techniques, and so on. The wireless connectivity can be based on the 802.11 family or “Wi-Fi™”, Bluetooth™, Zigbee™, and so on. The wireless connectivity can be based on near field communication (NFC). The wireless connectivity can be based on near field magnetic induction (NFMI). The wireless connectivity can be provided as part of a wireless personal area network (WPAN). In some embodiments, the wireless connectivity can be enabled by using a wireless transceiver to implement the desired wireless connectivity. In embodiments, the wireless communication channel enables bidirectional communication to an extraoral processor. For example, the speech generated by the person and sensed by the intraoral speech sensing complex can be sent to a wireless phone, laptop, tablet, or other wireless device. The speech can be converted to text for chat applications, word processors, and so on. The speech can be converted to commands to an operating system, file system, other applications, etc. The speech can be reconstituted by a receiving device and transmitted further, such as over a cellphone audio link, to enable silent speech to be received as normal audio speech. In some embodiments, the extraoral processor can provide additional speech sensing capabilities. For example, the data from the mouthpad 120 can be linked to video of the person from a cellphone, laptop, or video camera. The video data can be analyzed to provide additional machine learning model training input. The speech from the intraoral speech sensing complex can be used as input to a 3D video representation of a person so that as the person speaks, the speech sensed by the intraoral speech sensing complex generates words, mouth movements, jaw movements, and so on that are replicated by the 3D video image. The 3D video image can be used as part of a video chat, livestream, short-form video, etc.

The flow 100 includes sensing speech 150, based on an output of the processor. As mentioned above, an air soundwave microphone can enable normal speech sensing. In embodiments, the speech that is sensed can comprise silent speech. The silent speech 152 can comprise mouthing. In embodiments, the speech that is sensed can comprise low-volume speech. The low-volume speech 154 can comprise whispered speech. As mentioned above and throughout, data from all sensors included in the mouthpad are used to analyze and interpret the speech generated by the person. The speech analysis includes the machine learning model, so that the resulting words generated by the mouthpad processor include data from the sensors as recognized by the machine learning model. For example, the word “verbalize” can be spoken by the person as data input for the machine learning model. The mouthpad sensor data can include the tongue and jaw movement, the movement of air, the head and mouth positions, and the sound of the word in the training library and machine learning model. When the person says the word “verbalize” again, the associated tongue, jaw, and mouth movements, the movement of air, and the head position can be analyzed and compared to the machine learning model, even if the word is spoken silently. If the word is spoken in low-volume or at regular volume, microphone data can also be used in the analysis. The more mouthpad sensor data that matches or closely corresponds to the machine learning model data recorded for the word “verbalize”, the more likely the processor analysis will be to generate the word “verbalize” as its output. As more speech is generated by the person, and the person validates and corrects the processor output, the machine learning training library can continue to be updated and the machine learning model and processor output accuracy can continue to improve.

The flow 100 further comprises providing audio feedback 160 to the person, based on the analyzing. In embodiments, the audio feedback is enabled by a bone conduction speaker. A bone conduction speaker transmits sound to the inner ear through the bones of the skull rather than through the air via the car canal. The bone conduction speaker is attached to a tooth, or gums, or both tooth and gums in the oral cavity of the person. The detecting provides a digital output from the processor. The detecting further comprises transforming the digital output into an analog output before it is received by the bone conduction speaker. As speech is sensed by the mouthpad and analyzed by the processor, the resulting words from the processor are generated as digital output. The digital output can be converted into analog output and sent to the bone conduction speaker 162. The person can hear the words as they are spoken, even if the words are spoken in low volume or silently. In embodiments, the audio feedback comprises non-blocking audio feedback. Non-blocking audio feedback allows the person to continue to use the mouthpad as the audio feedback is being played by the speaker. Thus, the person can listen to the speech sensed and analyzed by the mouthpad as it is spoken. If a word is incorrectly analyzed or improperly pronounced, the person can use the mouthpad to pause the analysis and correct the word or mark the word for later review. Corrected words can be used to update the machine learning model and training library so that the processor continues to improve with time and usage.

Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors. Various embodiments of the flow 100, or portions thereof, can be included on a semiconductor chip and implemented in special purpose logic, programmable logic, and so on.

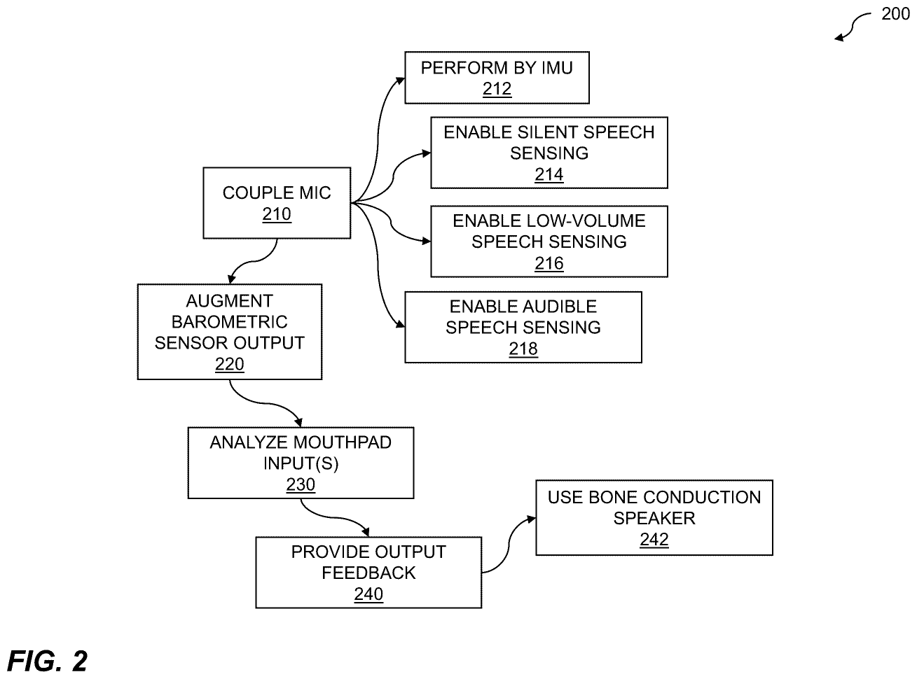

FIG. 2 is a flow diagram for intraoral speech sensing usage. The flow 200 includes a microphone 210 coupled to the intraoral speech sensing complex. The microphone 210 can include a pickup, a transducer, or another device that can be used to collect audio data within the oral cavity. The microphone can collect ambient sounds, speech, human-generated sounds, and so on. In embodiments, the microphone enables audible 218 and low-volume 216 speech sensing. The microphone can be used while the person (user) is in a situation where she or he is not able or permitted to speak out loud. In embodiments, the microphone can be enabled based on an output from an interface-embedded sensor, such as the TPS, the one or more IMUs, and/or other embedded sensors. The microphone can be operated using a “normally off” technique where the microphone can be enabled or turned on based on the TPS, IMU, or barometric sensors. In some embodiments, the coupling of the microphone is performed by the IMU sensor 212. IMU sensors can include microphones and bone conduction processes to receive bone-vibration audio data, which can also be sent, and send it to a bone conduction speaker 242 and output the sound data to the mousepad processor for analysis.

The flow 200 includes sensing speech, based on an output of the processor. The processor analyzes input from sensors included in the mouthpad, including a tongue position sensor (TPS), one or more inertial measurement unit (IMU) sensors, a barometric sensor, and the microphone 210. In embodiments, the speech that is sensed can include silent speech 214. The silent speech can include mouthing. In embodiments, the speech that is sensed can include low-volume speech 216. The low-volume speech can include whispered speech. Mousepad input data from the TPS, IMU sensors, and barometric sensors can all be used to identify speech as it occurs, regardless of the volume of sound produced by the person. In some embodiments, the person can prepare the mousepad for low-volume or silent speech by tapping the mousepad with the tongue. In embodiments, the speech that is sensed can include audible or normal speech. As mentioned above, the microphone 210 enables normal speech sensing. As the person speaks, the mousepad sensors are enabled and collect data from the various sensors. The data can be used to update the machine learning training library as well as being transmitted to the bone conduction speaker 242 and external wireless devices.

The flow 200 includes the microphone 210 augmenting output from the barometric sensor 220 for higher accuracy pressure change determination. As the person speaks, the output from the microphone 210 can be associated with the barometric sensor data 220 and stored in the machine learning model database. As the same words are spoken multiple times by the person, more barometric sensor data can be accumulated and associated with the words so that when the words are spoken with little or no sound, the barometric sensor data can be more accurately matched to the correct words. In other embodiments, the same association of microphone data to sensor data can be rendered for the IMU and TPS sensors. Thus, the ability of the mousepad to associate the correct words with sensor data can improve, even when the words are spoken with low or no volume.

The flow 200 includes analyzing input from the mouthpad 230, wherein the analyzing is performed on a processor coupled to the mouthpad. In embodiments, the speech analyzing includes a machine learning model, so that the resulting words generated by the mouthpad processor include data from the sensors as recognized by the machine learning model. As words are spoken by the person, the mouthpad sensor can collect information including the tongue and jaw movement, the movement of air, the head and mouth positions, and the sound of the word itself. This data can be included in the training library and machine learning model. When the person says the same word again later, the associated tongue, jaw, and mouth movements, the movement of air, and the head position can be analyzed and compared to the machine learning model, even if the word is spoken silently. If the word is spoken in low-volume or at regular volume, audio microphone data can also be used in the analysis. The more mouthpad sensor data that matches or closely corresponds to the machine learning model data recorded for each word, the more likely the processor analysis will be to generate the correct word as its output. As more speech is generated by the person, and the person validates and corrects the processor output, the machine learning training library can continue to be updated and the machine learning model and processor output accuracy can continue to improve.

In embodiments, the tongue movement of the person that is detected comprises continuous sensing of the tongue along the surface of the mouthpad. The continuous sensing can include three-dimensional sensing. The TPS can be in an “always on” mode when the mouthpad is embedded in the oral cavity of the person. The position of the tongue within the oral cavity is continually tracked by the TPS and IMU sensors. The combination of TPS and IMU sensors can allow tongue location information to be gathered along all positional axes. In embodiments, the mouthpad can include pressure sensors, optical sensors, ultrasonic sensors, etc. Input from the tongue on the mouthpad can be detected and used to augment, control, or modify speech data as it is collected by the sensors and microphones in the oral cavity. In some embodiments, the person can use the mouthpad to control the speaker, including adjusting the volume, shutting the speaker on and off, etc. The mouthpad can be used to control the wireless connection to external devices, such as a cellphone, computer, or other networked device.

The flow 200 includes providing audio feedback 240 to the person, based on the analyzing. In embodiments, the audio feedback is enabled by a bone conduction speaker. The bone conduction speaker is attached to a tooth, or gums, or both tooth and gums in the oral cavity of the person. In embodiments, the microphone and processor both generate digital output signals. The output feedback further comprises transforming the digital output into an analog output before it is received by the bone conduction speaker 242. The analog signal received by the bone conduction speaker allows the person to hear the words sensed by the mousepad as they are spoken, even if the words are spoken in low volume or silently. In embodiments, the audio feedback comprises non-blocking audio feedback. Non-blocking audio feedback allows the person to continue to use the mouthpad as the audio feedback is being played by the speaker. Thus, the person can listen to the speech sensed and analyzed by the mouthpad as it is spoken. If a word is analyzed incorrectly or improperly pronounced, the person can use the mouthpad to pause the analysis and correct the word or mark the word for later review. Corrected words can be used to update the machine learning model and training library so that the processor continues to improve with time and usage.

Various steps in the flow 200 may be changed in order, repeated, omitted, or the like without departing from the disclosed concepts. Various embodiments of the flow 200 can be included in a computer program product embodied in a non-transitory computer readable medium that includes code executable by one or more processors. Various embodiments of the flow 200, or portions thereof, can be included on a semiconductor chip and implemented in special purpose logic, programmable logic, and so on.

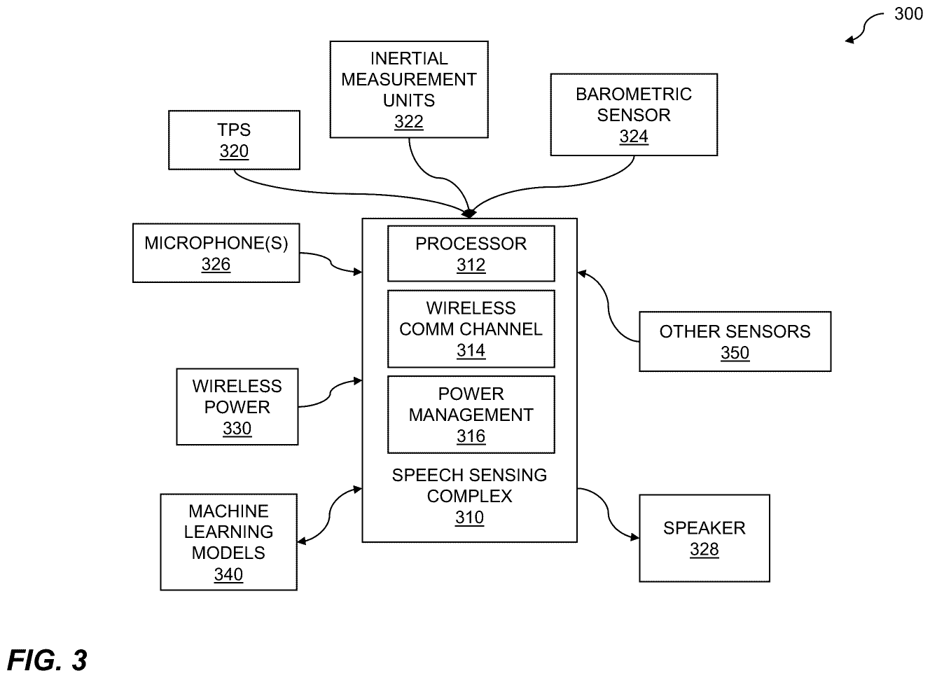

FIG. 3 is a system block diagram for silent intraoral speech sensing. The diagram 300 includes an intraoral speech sensing complex 310. The intraoral speech sensing complex 310 includes a processor 312. The processor 312 analyzes input from sensors included in a mouthpad which is embedded in an oral cavity of a person. In embodiments, the sensors include a tongue position sensor (TPS). The TPS 320 can include electrodes, pressure sensors, barometric sensors, optical sensors, ultrasonic sensors, etc. Tongue input data can be detected by the TPS and can augment, control, or modify data collected by, or being processed by, a processor or preprocessor. The tongue input data can be sensed based on tongue position, tongue pressure, tongue movement, tongue movement direction, tongue movement speed, tongue movement acceleration, and so on.

In embodiments, the sensors include a barometric sensor. The barometric sensor 324 can be used to detect ambient barometric pressure, changes in barometric pressure such as increases or decreases, and so on. The barometric sensor can detect a tri-value state of air pressure within an oral cavity containing the interface. The tri-value state can include a number, a code, a percentage, text, and the like. The tri-value state can be sensed from a continuum of pressures and can include ambient barometric pressure, increased barometric pressure due to exhaling into a closed oral cavity, and decreased barometric pressure due to inhaling from a closed oral cavity. The barometric sensor 324 can detect a plurality of pressures along an analog or continuum of possible pressures for sensing.

In embodiments, the sensors include one or more inertial measurement units (IMUs). The IMUs 322 can be used to detect tongue or jaw positions, rotation, acceleration, and so on. The IMUs can be placed at various positions within the mouth. In some embodiments, at least two inertial measurement units are located in nonadjacent locations within the mouth. An example placement of the IMUs is where the IMUs are located to the left and the right of a retainer. In other embodiments, the inertial measurement unit is adjacent to the palate of the mouth. The positioning of the IMU can be determined based on efficacy, comfort, convenience, and so on.

The diagram 300 includes a microphone 326. In embodiments, the microphone is coupled to the intraoral speech sensing complex. The microphone 326 can include a pickup, a transducer, or another device that can be used to collect audio data within the oral cavity. In some embodiments, the microphone is coupled to the intraoral speech sensing complex by the IMU sensor. The microphone can collect ambient sounds, speech, human-generated sounds, and so on. In embodiments, the microphone can be enabled based on an output from an interface-embedded sensor, such as the TPS, the one or more IMUs, and/or other embedded sensors. The microphone can be operated using a “normally off” technique where the microphone can be enabled or turned on based on the TPS, IMUs, or barometric sensors. In other embodiments, the microphone can be enabled based on an output from another interface-embedded sensor 350.

The diagram 300 includes a wireless communication channel 314 which is coupled to the intraoral speech sensing complex 310. The wireless connectivity can be based on communications standards, preferred protocols, low power techniques, and so on. In some embodiments, the wireless connectivity can be enabled by using a wireless transceiver to implement the desired wireless connectivity. The wireless communication channel enables bidirectional communication to an extraoral processor. For example, the speech generated by the person and sensed by the intraoral speech sensing complex can be sent to a wireless phone, laptop, tablet, or other wireless device. The speech can be converted to text for chat applications, word processors, and so on. The speech can be converted to commands to an operating system, file system, other applications, etc. In some embodiments, the extraoral processor can provide additional speech sensing capabilities. For example, the data from the mouthpad can be linked to video of the person from a cellphone, laptop, or video camera. The video data can be analyzed to provide additional machine learning model training input. The speech from the intraoral speech sensing complex can be used as input to a 3D video representation of a person so that as the person speaks, the speech sensed by the intraoral speech sensing complex generates words, mouth movements, jaw movements, and so on that are replicated by the 3D video image. The 3D video image can be used as part of a video chat, livestream, short-form video, etc.

The diagram 300 includes power management 316 for the intraoral speech sensing complex 310. The intraoral speech sensing complex is powered using a wireless power system 330. The wireless power system 330 comprises a battery, an induction coil, a battery charger, a power regulator, and a Hall sensor. The battery, induction coil, power regulator, and Hall sensor can be included in the intraoral speech sensing complex 310. In embodiments, the battery is charged using an external battery charger to induce voltage in the induction coil. The battery charger also includes an induction coil. When the induction coil in the external charger is brought into proximity with the induction coil in the intraoral speech sensing complex, electrical current is induced in the complex coil. The Hall sensor can be used to detect when the intraoral speech sensing complex has been placed in or near the battery charger. The Hall sensor can be used to control a switch in the speech sensing complex to allow the induced current to flow from the coil into the battery, thus charging it. The power regulator is used to maintain voltage from the battery to the components of the intraoral speech sensing complex as it operates inside the oral cavity of the person.

The diagram 300 includes a bone conduction speaker 328. The bone conduction speaker 328 can be used to enable audio feedback to the person, based on the analyzing done by the processor 312. In embodiments, the bone conduction speaker is attached to a tooth, or gums, or both tooth and gums in the oral cavity of the person. A bone conduction speaker transmits sound to the inner ear through the bones of the skull rather than through the air via the car canal. The microphone 326 and processor 312 both generate digital output signals. The digital output signals are transformed by an ultra-low power audio codec that is coupled to the intraoral speech sensing complex 310. An audio codec is a device capable of encoding or decoding a digital data stream which encodes or decodes audio data signals. The codec can receive input from the microphone 326 and the processor 312 and transform the input signals into analog output before they are received by the bone conduction speaker 328. The analog signal received by the bone conduction speaker allows the person to hear words sensed by the intraoral speech sensing complex 310 as they are spoken, even if the words are spoken in low volume or silently. In addition, the bone conduction speaker can be used to present audio feedback to the person from sources other than the person's low-volume or silent speech. The bone conduction speaker can receive input from processor 312 that originates from a sensor within the speech sensing complex 310. For example, if one of the other sensors 350 is a body temperature sensor, the processor can periodically send the person's temperature to the person using the bone conduction speaker. Any number of biometric sensors could be employed to help the person monitor his or her body vital signs throughout the day and/or night. Further, the processor can receive wireless input from an external device, such as a cellphone or a computer system, and process that input for audio presentation to the person. The input can be anything that comes from the phone or computing system that can be consumed in an audio format, such as a voice call, a text message readout, an email readout, an application-generated summary, a map direction, a procedure/instruction direction, an alert, a screen reader output, a response from an artificial intelligence agent, and so on.

The diagram 300 includes machine learning models 340. In embodiments, the machine learning models are used by the processor to associate data coming from the TPS, IMUs, barometric sensor, microphone, and other sensors with words stored in the machine learning model database. The combination of sensors and machine learning models 340 allow the intraoral speech sensing complex 310 to recognize words that are spoken in low volume or silently by the person. The sensors, in combination with the microphone and the machine learning models, allow words spoken with normal volume to be recognized as well. Thus, words spoken at any volume level, from silent to full volume, can be detected and analyzed by the intraoral speech sensing complex and output to the person through the bone conduction speaker. The wireless communication channel 314 allows the words, or commands generated by the person through the mouthpad, to be sent to external devices, such as a cellphone, laptop, tablet, computer, or other networked devices.

In embodiments, the diagram comprises an apparatus for speech sensing comprising: an embedded tongue position sensor (TPS) in an oral cavity of a person, wherein the TPS is embedded palatally in the oral cavity, and wherein the TPS detects tongue movement of the person; a barometric sensor and an inertial measurement unit (IMU) sensor coupled to the TPS, wherein the barometric sensor and the IMU sensor are both located in the oral cavity of the person, and wherein the TPS, the barometric sensor, and the IMU sensor comprise a mouthpad; and a processor coupled to the mouthpad, wherein the processor analyzes input from the mouthpad, wherein the processor and the mouthpad comprise an intraoral speech sensing complex, and wherein an output channel from the processor delivers sensed speech of the person.

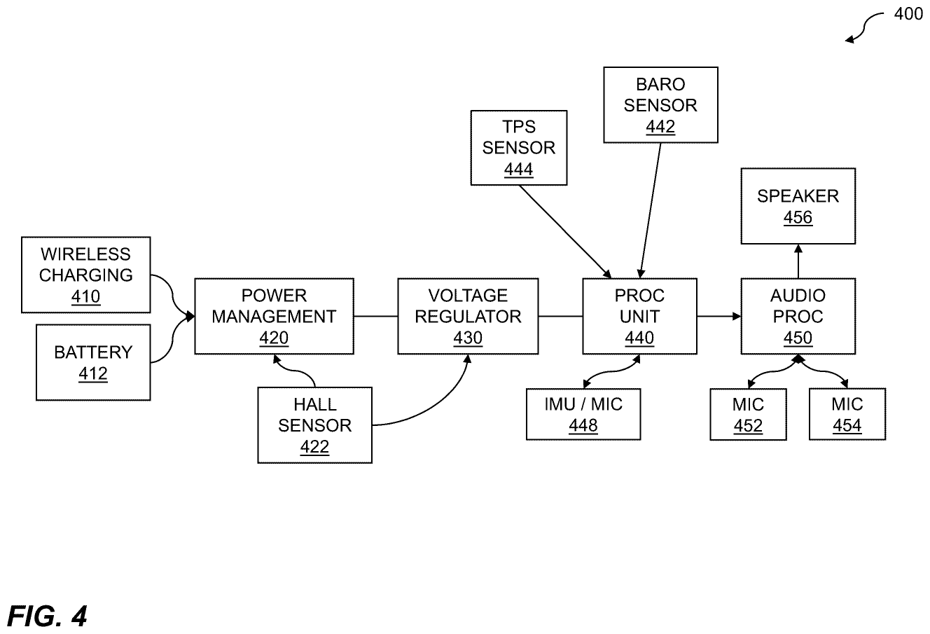

FIG. 4 illustrates system components for silent intraoral speech sensing. The illustration 400 includes a power management system 420. In embodiments, the power management system is coupled to an induction coil for wireless charging, a battery, a wireless internal battery charger, a voltage regulator, and a Hall sensor. The battery 412, induction coil 410, voltage regulator 430, power management system 420, and Hall sensor 422 are included in the intraoral speech sensing complex. In embodiments, the battery is charged using an external battery charger to induce voltage in the induction coil. The external battery charger also includes an induction coil. When the induction coil in the external charger is brought into proximity with the induction coil 410 in the intraoral speech sensing complex, electrical current is induced in the internal coil. The Hall sensor 422 can be used to detect when the intraoral speech sensing complex has been placed in or near the external battery charger. The Hall sensor 422 can be used to control the internal battery charger in the speech sensing complex and allow the induced current to flow from the induction coil 410 into the battery 412, thus charging it. In some embodiments, the Hall sensor can be used to disable the voltage regulator and inhibit power from flowing to components of the intraoral speech sensing complex while battery charging is underway. The voltage regulator 430 is used to maintain voltage from the battery 412 to the components of the intraoral speech sensing complex as it operates inside the oral cavity of the person.

The illustration 400 includes a processor unit 440. The processor unit 440 analyzes input from sensors included in a mouthpad which is embedded in an oral cavity of a person. The sensors which comprise the mouthpad, along with the processor 440, include a tongue position sensor (TPS) 444 which detects tongue movement of the person, a barometric sensor 442 which detects breathing and air pressure inside the oral cavity, and one or more inertial measurement sensors (IMUs) 448 which detect jaw and/or head movements. In some embodiments, the IMUs 448 can be combined with one or more microphones. In other embodiments, one or more microphones, such as microphones 452 and 454 can be separate from the IMUs. The processor unit 440 analyzes data from the sensors and microphones with machine learning models to associate words spoken or mouthed by a person with words stored in the machine learning model. The processor unit 440 includes a machine learning training library which allows the machine learning models to be updated and improved as words are validated and corrected by the person. The combination of sensors and machine learning models allow the intraoral speech sensing complex to recognize words that are spoken in low volume or silently by the person. The sensors, in combination with the microphones and the machine learning models, allow words spoken with normal volume to be recognized as well. Thus, words spoken at any volume level, from silent to full volume, can be detected and analyzed by the intraoral speech sensing complex and can be output to the person through a bone conduction speaker 456.

The illustration 400 includes an audio processor 450. The audio processor 450 is comprised of an ultra-low power audio codec that is included in the intraoral speech sensing complex. An audio codec is a device capable of encoding or decoding a digital data stream which encodes or decodes audio data signals. The codec can receive input from the microphones 452, 454 and the processor unit 440 and transform the input signals into analog output before they are sent to the bone conduction speaker 456. The bone conduction speaker 456 can be used to enable audio feedback to the person, based on the analyzing done by the processor unit 440. In embodiments, the bone conduction speaker is attached to a tooth, or gums, or both tooth and gums in the oral cavity of the person. A bone conduction speaker transmits sound to the inner ear through the bones of the skull rather than through the air via the car canal. The microphones 452, 454 and processor unit 440 generate digital output signals. The digital output signals are transformed by the audio processor 450 to analog signals. The analog signals received by the bone conduction speaker 456 allow the person to hear words sensed by the intraoral speech sensing complex as they are spoken, even if the words are spoken in low volume or silently.

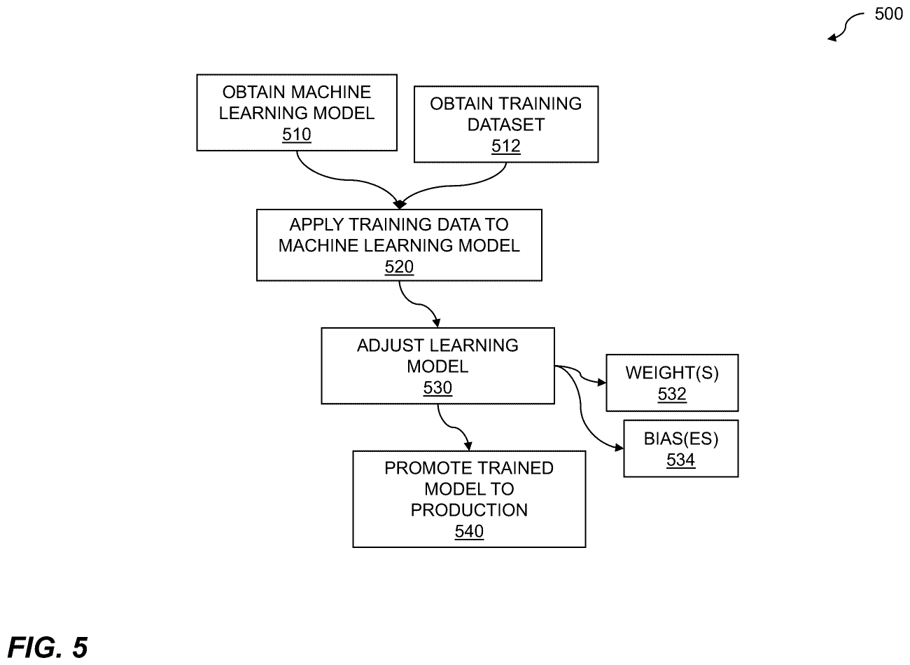

FIG. 5 is a flow diagram for machine learning. The flow 500 includes obtaining a machine learning model 510. A machine learning model is a computer program that can learn from data and make decisions based on patterns it identifies in the data. The machine learning model is created using one or more algorithms, which are rules that tell the computer how to learn from data. In embodiments, the machine learning model used by the processing unit included in the intraoral speech sensing complex can be composed of data from tongue position sensors (TPSs), barometric sensors, inertial measurement sensors (IMUs), and microphones, combined with words. Each word in the machine learning model can be associated with data from the sensors and microphones. The objective is to use the machine learning model to identify words from the model database based on data obtained from sensors and microphones.

The flow 500 includes obtaining a training dataset 512. The training dataset 512 is used to train the machine learning model to analyze data and recognize words based on input from the sensors and microphones included in the intraoral speech sensing complex. The training dataset 512 can be obtained from various sources, including academic institutions, research facilities, government websites, corporate facilities, manufacturers, and so on. In embodiments, training dataset sources can include sensor manufacturers, audio engineering websites, natural language processing (NLP) development facilities, and so on. In embodiments, more than one training dataset can be used to generate ranges of sensor and microphone data that match specific words stored by the machine language model.

The flow 500 includes applying the training data 512 to the machine learning model 520. Training the machine learning model involves several steps. First, real data is collected, cleaned, and stored in the model. In some embodiments, a predetermined set of words and phrases can be spoken by the person using the intraoral speech sensing complex. As known words and phrases are spoken by the person, the associated sensor and microphone data is recorded and stored in the model. Each word can be spoken with different volume levels, from loud to silent, shouted, whispered, or murmured. Words can be spoken or mouthed at different speeds, different pitches, and so on. Each set of sensor and microphone data associated with a word in the model can be used to create patterns that can be recognized by the machine learning model in future iterations. After the predetermined words have been stored to the model, words and phrases from the training data can be used to teach the machine learning model to identify words from the sensor and microphone data included in the training data. As data is analyzed by the machine learning model using a machine learning algorithm, the model generates a prediction for each input word from the training dataset. The prediction is scored regarding whether or not the correct word was identified by the model. There are many machine learning model algorithms available from many public and private sources. As the training data is applied to the machine learning model 520, different algorithms can be tried to adjust the learning model 530 so that it identifies words more quickly and accurately.

The flow 500 includes adjusting the machine learning model 530. Along with using different machine learning algorithms to adjust the model, machine learning model weights 532 and biases 534 can be analyzed and used. Machine learning model weights 532 are used to describe the importance of each feature in a model algorithm. The higher the weight, the more important the feature is in determining the outcome of the algorithm. Weights can be positive or negative in value and can be assigned to any type of data included in the machine learning model. For example, each word in the machine learning model for the intraoral speech sensing complex can include microphone, IMU, TPS, and barometric sensor data. Weights can be assigned to each sensor and the microphone. The weights can be assigned so that microphone data is given a +2 weight, while TPS data is given a +1 weight, and IMU data is assigned a weight of 0. As additional training passes are made, the weights in the model algorithm can be adjusted to favor sensor data more or less strongly in order to achieve more accurate word identification. As multiple training passes are conducted, the machine learning model can determine which weight settings are the most effective in identifying words correctly.

Machine learning model biases 534 are phenomena that develop when a machine learning model produces results that are systematically prejudiced against certain groups or types of data. Biases 534 can occur when the training data used to develop a machine learning model is biased or when a machine learning algorithm tends to favor one type of data over others. For example, the model can exhibit a bias toward midwestern or western pronunciations of words based on the training data used to build and refine the model. In order to adjust the machine learning model for use in a broader population, training data using words spoken by people from different sections of the country can be included. English words spoken by people from different countries can be used as well in order to further expand the range of sensor and microphone data associated with each word in the model.

The flow 500 includes promoting the trained model to production 540. Once the machine learning model has achieved an acceptable level of accuracy in identifying words input from the training datasets, the model can be placed into production 540. In an example, the machine learning model and the training dataset can be loaded into the intraoral speech sensing complex. Even in production, the machine learning model can continue to learn and improve its ability to predict words accurately. The person wearing the mouthpad can hear each word analyzed by the processor and indicate whether or not any word has been incorrectly identified. In embodiments, the person can correct the model immediately by indicating an error using the mouthpad, then respeaking the word to be corrected. In some embodiments, the person can mark the word for later review. In other embodiments, the words analyzed by the processor can be sent to a chat or text application on a cellphone or computer through the wireless connection included in the complex. The person can mark incorrect words, then spell out the correct word in the application and send it back to the processor. The update can be added to the training dataset and to the machine learning model. As the person continues to validate and correct the machine learning model, the model becomes more and more accurate.

Various steps in the flow 500 may be changed in order, repeated, omitted, or the like without departing from the disclosed concepts. Various embodiments of the flow 500 can be included in a computer program product embodied in a non-transitory computer readable medium that includes code executable by one or more processors. Various embodiments of the flow 500, or portions thereof, can be included on a semiconductor chip and implemented in special purpose logic, programmable logic, and so on

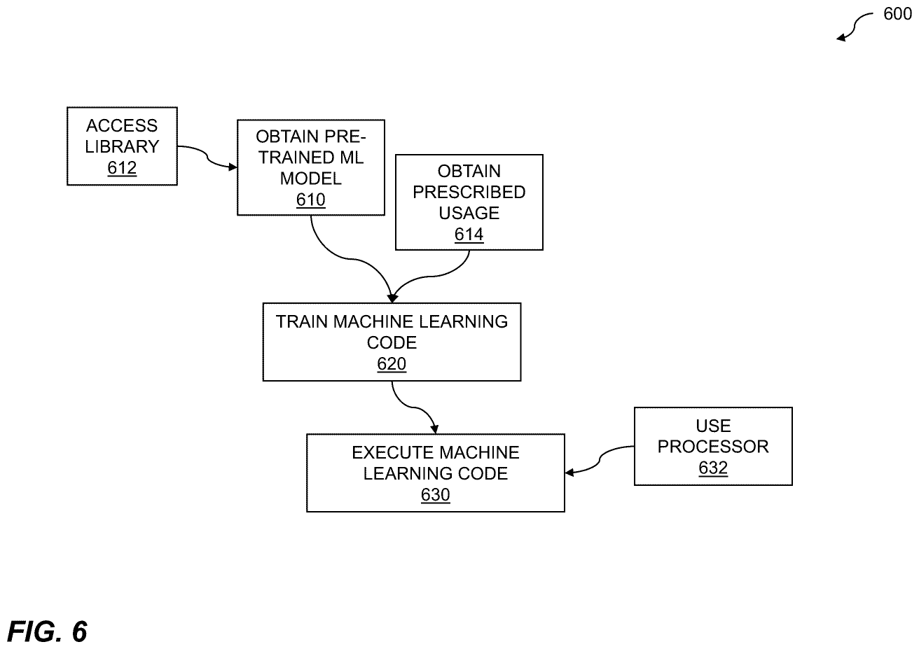

FIG. 6 is a flow diagram for machine learning training. The flow 600 includes obtaining a pretrained machine learning model 610. Pretrained machine learning models can be obtained from many sources including private and public websites, corporate websites, application developers, manufacturers, and so on. In embodiments, a pretrained machine learning model can be obtained from corporate or departmental sources, natural language processing vendors or developers, speech therapy sources, and so on. Libraries of machine learning models and related data can be accessed 612 from internal or external sources. Training datasets that are loaded and filtered for the prescribed usage 614 of the intraoral speech sensing complex can be obtained. The training datasets can initially include data that is not relevant to the speech sensing complex usage. For instance, word definitions, synonyms, and antonyms can be included along with a dictionary of English words read aloud by a professional narrator. The unneeded data can be filtered out so that the training data set feeds the machine language model only what is necessary.

The flow 600 includes training the machine learning code 620. In embodiments, training data from the prescribed usage dataset is used as input to the processor 632 included in the intraoral speech sensing complex. The machine learning model can also be loaded into the intraoral speech sensing complex. The processor takes in the training data and executes the machine learning code 630. As the training data is analyzed by the machine learning code 630 using a machine learning algorithm, the model generates a prediction for each input word from the training dataset. The predicted word is sent by the processor 632 as digital output to a bone conduction speaker and/or an external wireless device. The prediction is scored as to whether the correct word was identified by the model. If the word is predicted correctly, any variations in the training data are added to the machine learning model data entry for the word. If the prediction is incorrect, the correct word is identified by the person and the training data for the correct word can be added to the machine learning model. In some embodiments, adjustments to the weights related to the machine learning model can be made based on the accuracy of the model across various words and word groups. As training continues, the accuracy of the machine learning model continues to improve.

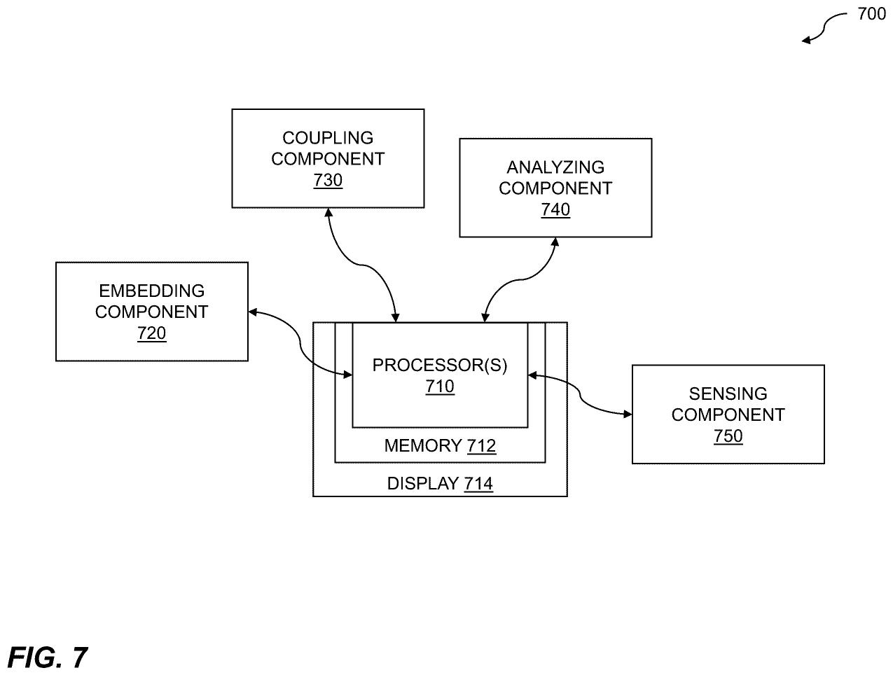

FIG. 7 is a system diagram for silent intraoral speech sensing with audio feedback. The system 700 includes one or more processors 710 coupled to a memory 712 which stores instructions. The system 700 includes a display 714 coupled to the one or more processors 710 for displaying data, database information, programming details, intermediate steps, instructions, and so on. In embodiments, one or more processors 710 are coupled to the memory 712 where the one or more processors, when executing the instructions which are stored, are configured to: access a tongue position sensor (TPS) in an oral cavity of a person, wherein the TPS is embedded palatally in the oral cavity, wherein the TPS detects tongue movement of the person; wherein a barometric sensor and an inertial measurement unit (IMU) sensor are coupled to the TPS, wherein the barometric sensor and the IMU sensor are both located in the oral cavity of the person, wherein the TPS, the barometric sensor, and the IMU sensor comprise an intraoral mouthpad, and wherein the mouthpad detects tongue movements, jaw and/or head movements, and breathing of the person; analyze input from the mouthpad, wherein the analyzing is performed on a processor coupled to the mouthpad, and wherein the processor and the mouthpad comprise an intraoral speech sensing complex; and sense speech, based on an output of the processor.

The system 700 includes an embedding component 720. The embedding component 720 includes functions and instructions for embedding a tongue position sensor (TPS) in an oral cavity of a person, wherein the TPS is embedded palatally in the oral cavity, and wherein the TPS detects tongue movement of the person. In embodiments, the tongue movement of the person that is detected comprises continuous sensing of the tongue along the surface of the mouthpad. The continuous sensing includes three-dimensional sensing. The TPS can be in an “always on” mode when the mouthpad is embedded in the oral cavity of the person. Input from the tongue on the mouthpad can be detected and used to augment, control, or modify speech data as it is collected by the sensors and microphones in the oral cavity. In some embodiments, the person can use the mouthpad to control the speaker, including adjusting the volume, shutting the speaker on and off, etc. The mouthpad can be used to control the wireless connection to external devices, such as a cellphone, computer, or other networked device.

The system 700 includes a coupling component 730. The coupling component 730 includes functions and instructions for coupling a barometric sensor and an inertial measurement unit (IMU) sensor to the TPS, wherein the barometric sensor and the IMU sensor are both located in the oral cavity of the person, wherein the TPS, the barometric sensor, and the IMU sensor comprise an intraoral mouthpad, and wherein the mouthpad detects tongue movements, jaw and/or head movements, and breathing of the person. In embodiments, the coupling includes a microphone to the intraoral speech sensing complex. In some embodiments, the coupling of the microphone can be performed by the IMU sensor. The coupling includes a wireless communication channel to the intraoral speech sensing complex. The wireless communication channel enables bidirectional communication to an extraoral processor. The extraoral processor can provide additional speech sensing capabilities.

The coupling component 730 includes powering the intraoral speech sensing complex using a wireless power system. The wireless power system comprises a battery, an induction coil, a battery charger, a power regulator, and a Hall sensor. The battery, induction coil, power regulator, wireless internal battery charger, and Hall sensor are included in the intraoral speech sensing complex. In embodiments, the battery is charged using an external battery charger to induce voltage in the induction coil. The external battery charger also includes an induction coil. When the induction coil in the external charger is brought into proximity with the induction coil in the intraoral speech sensing complex, electrical current is induced in the internal coil. The Hall sensor can be used to detect when the intraoral speech sensing complex has been placed in or near the external battery charger. The Hall sensor can be used to control the internal battery charger in the speech sensing complex and allow the induced current to flow from the induction coil into the battery, thus charging it. In some embodiments, the Hall sensor can be used to disable the voltage regulator and inhibit power from flowing to components of the intraoral speech sensing complex while battery charging is underway. The voltage regulator is used to maintain voltage from the battery to the components of the intraoral speech sensing complex as it operates inside the oral cavity of the person. The coupling component 730 includes providing audio feedback to the person. The audio feedback is enabled by a bone conduction speaker. The bone conduction speaker is attached to a tooth, or gums, or both tooth and gums in the oral cavity of the person.

The system 700 includes an analyzing component 740. The analyzing component 740 includes functions and instructions for analyzing input from the mouthpad, wherein the analyzing is performed on a processor coupled to the mouthpad, and wherein the processor and the mouthpad comprise an intraoral speech sensing complex. In embodiments, the mouthpad includes data from the TPS, the barometric sensor, the IMUs, and the microphones. The processor analyzes the data from the mouthpad to sense and interpret speech generated by the person. The position of the tongue, mouth, and jaw can be used to clarify and validate the words spoken by the person. The incoming and outgoing breath of the person, and the pressures generated by the air flowing in and out of the mouth, can be used to analyze the words, inflections, accent, intensity, and so on of the speech being generated.

The system 700 includes a sensing component 750. The sensing component 750 includes functions and instructions for sensing speech, based on an output of the processor. In embodiments, the speech that is sensed can include normal speech sensing. The speech that is sensed comprises silent speech, wherein the silent speech comprises mouthing. The speech that is sensed comprises low-volume speech, wherein the low-volume speech comprises whispered speech. Data from all sensors included in the mouthpad are used to analyze and interpret the speech generated by the person. The speech analysis includes the machine learning model, so that the resulting words generated by the mouthpad processor include data from the sensors as recognized by the machine learning model. The mouthpad sensor data can include the tongue and jaw movement, the movement of air, the head and mouth positions, and the sound of the word in the training library and machine learning model.

The system 700 can include a computer program product in a non-transitory computer readable medium for speech sensing, the computer program product comprising code which causes one or more processors to perform operations of: embedding a tongue position sensor (TPS) in an oral cavity of a person, wherein the TPS is embedded palatally in the oral cavity, and wherein the TPS detects tongue movement of the person; coupling a barometric sensor and an inertial measurement unit (IMU) sensor to the TPS, wherein the barometric sensor and the IMU sensor are both located in the oral cavity of the person, wherein the TPS, the barometric sensor, and the IMU sensor comprise an intraoral mouthpad, and wherein the mouthpad detects tongue movements, jaw and/or head movements, and breathing of the person; analyzing input from the mouthpad, wherein the analyzing is performed on a processor coupled to the mouthpad, and wherein the processor and the mouthpad comprise an intraoral speech sensing complex; and sensing speech, based on an output of the processor.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited to neither conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM); an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States, then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for speech sensing comprising:
embedding a tongue position sensor (TPS) in an oral cavity of a person, wherein the TPS is embedded palatally in the oral cavity, and wherein the TPS detects tongue movement of the person;
coupling a barometric sensor and an inertial measurement unit (IMU) sensor to the TPS, wherein the barometric sensor and the IMU sensor are both located in the oral cavity of the person, wherein the TPS, the barometric sensor, and the IMU sensor comprise an intraoral mouthpad, and wherein the mouthpad detects tongue movements, jaw and/or head movements, and breathing of the person;
analyzing input from the mouthpad, wherein the analyzing is performed on a processor coupled to the mouthpad, and wherein the processor and the mouthpad comprise an intraoral speech sensing complex; and
sensing speech, based on an output of the processor.

2. The method of claim 1 wherein the tongue movement of the person that is detected comprises continuous sensing of the tongue along a surface of the mouthpad.

3. The method of claim 2 wherein the continuous sensing includes three-dimensional sensing.

4. The method of claim 1 wherein the speech that is sensed comprises silent speech.

5. The method of claim 4 wherein the silent speech comprises mouthing.

6. The method of claim 1 wherein the speech that is sensed comprises low-volume speech.

7. The method of claim 6 wherein the low-volume speech comprises whispered speech.

8. The method of claim 1 further comprising providing audio feedback to the person, based on the analyzing.

9. The method of claim 8 wherein the audio feedback is enabled by a bone conduction speaker.

10. The method of claim 9 wherein the bone conduction speaker is attached to a tooth, or gums, or both tooth and gums in the oral cavity of the person.

11. The method of claim 9 wherein the detecting provides a digital output from the processor.

12. The method of claim 11 further comprising transforming the digital output into an analog output before it is received by the bone conduction speaker.

13. The method of claim 8 wherein the audio feedback comprises non-blocking audio feedback.

14. The method of claim 1 further comprising coupling a microphone to the intraoral speech sensing complex.

15. The method of claim 14 wherein the coupling of the microphone is performed by the IMU sensor.

16. The method of claim 14 wherein the microphone augments output from the barometric sensor for higher accuracy pressure change determination.

17. The method of claim 14 wherein the microphone enables normal speech sensing.

18. The method of claim 1 further comprising powering the intraoral speech sensing complex using a wireless power system.

19. The method of claim 18 wherein the wireless power system comprises a battery, an induction coil, a battery charger, a power regulator, and a Hall sensor.

20. The method of claim 1 wherein the processor executes machine learning code.

21. The method of claim 20 further comprising training the machine learning code.

22. The method of claim 21 wherein the training is based on prescribed usage of the intraoral speech sensing complex by the person.

23. The method of claim 1 further comprising coupling a wireless communication channel to the intraoral speech sensing complex.

24. The method of claim 23 wherein the wireless communication channel enables bidirectional communication to an extraoral processor.

25. An apparatus for speech sensing comprising:
an embedded tongue position sensor (TPS), wherein the TPS is suitable for embedding palatally within an oral cavity of a person, and wherein the TPS detects tongue movement of the person;
a barometric sensor and an inertial measurement unit (IMU) sensor coupled to the TPS, wherein the barometric sensor and the IMU sensor are both located in the oral cavity of the person, and wherein the TPS, the barometric sensor, and the IMU sensor comprise a mouthpad; and
a processor coupled to the mouthpad, wherein the processor analyzes input from the mouthpad, wherein the processor and the mouthpad comprise an intraoral speech sensing complex, and wherein an output channel from the processor delivers sensed speech of the person.

26. A computer system for speech sensing comprising:
a memory which stores instructions;
one or more processors coupled to the memory wherein the one or more processors, when executing the instructions which are stored, are configured to:
access a tongue position sensor (TPS) in an oral cavity of a person, wherein the TPS is embedded palatally in the oral cavity, wherein the TPS detects tongue movement of the person, wherein a barometric sensor and an inertial measurement unit (IMU) sensor are coupled to the TPS, wherein the barometric sensor and the IMU sensor are both located in the oral cavity of the person, wherein the TPS, the barometric sensor, and the IMU sensor comprise an intraoral mouthpad, and wherein the mouthpad detects tongue movements, jaw and/or head movements, and breathing of the person;
analyze input from the mouthpad, wherein the analyzing is performed on a processor coupled to the mouthpad, and wherein the processor and the mouthpad comprise an intraoral speech sensing complex; and
sense speech, based on an output of the processor.
